(19) United States
(12) Patent Application Publication
(10) Pub. No.: US 2014/0336615 A1
(43) Pub. Date: Nov. 13, 2014

(54) Systems And Methods For Injecting Cellular Fluids

(71) Applicant: Bayer Medical Care Inc., Indianola, PA (US)

(72) Inventors: Martin J. Uram, Pittsburgh, PA (US); Paul D. Levin, Pittsburgh, PA (US); Frederick W. Trombley, III, Gibsonia, PA (US); Mark Trocki, Cheswick, PA (US)

(21) Appl. No.: 14/361,357

(22) PCT Filed: Nov. 28, 2012
(86) PCT No.: PCT/US2012/066792

§ 371 Date: May 29, 2014

Related U.S. Application Data
(60) Provisional application No. 61/566,139, filed on Dec. 2, 2011.

(51) Int. Cl. A61M 5/142; A61M 5/172

(52) U.S. Cl. CPC A61M 5/142; A61M 5/172; A61M 2005/14292; A61M 2005/14208; A61M 2202/09; A61M 2205/50; A61M 2205/3372
USPC 604/506; 604/67

(57) ABSTRACT

A system includes at least one pressurizing mechanism, a fluid path adapted to be placed in operative connection with the pressurizing mechanism to deliver an injectate to a patient, wherein the injectate includes cells, a control system operably associated with the at least one pressurizing mechanism; and at least one parameter generation system in operative connection with the control system. The parameter generation system includes an input system to receive data of a type of cells to be injected and is adapted to generate at least one parameter for the injection procedure at least in part on the basis of the data of the type of cells. The at least one parameter for the injection procedure may, for example, be a variable associated with the injectate, a variable associated with an injection protocol or a variable associated with at least one component of the fluid path.

### CROSS-REFERENCE TO RELATED APPLICATIONS

This application claims the benefit of U.S. Provisional Application No. 61/566,139, filed Dec. 2, 2011 and entitled “Systems and Methods for Injecting Cellular Fluids”, and which is incorporated herein in its entirety.

### STATEMENT OF GOVERNMENT INTEREST

The invention described herein was made in the performance of work under NASA Contract No. NNJ06HE87G and is subject to the provisions of Section 305 of the National Aeronautics and Space Act of 1958 (42 U.S.C. 2457). As such, the United States government has certain rights in this invention.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Cellular-based therapeutic agents or materials (sometimes referred to herein as cellular therapeutics) have received significant scientific and clinical interest in their potential to resolve a potentially large number of medical disorders. Such clinical interest has spanned the range of medical conditions including cardiac infarct, venous thromboses, type 1 diabetes, and various neurodegenerative diseases including Parkinson's and Alzheimer's disease. While there are a multitude of clinical procedures for these and other medical conditions, many of them are invasive, require significant time, expense, and physician training for operating room procedures, require significant in-hospital treatment and recovery time for the patient, and can have significant cost and risks associated with the procedures. The hope with a cellular therapeutic approach is that a relatively simple injection or infusion procedure of a target organ with an appropriate cellular therapeutic may reduce significantly all of these factors.

Several biologic, including cellular, therapeutics have been considered in recent years. Representative examples include a suspension of natural or modified cells, a suspension of modified viral particles, or viruses or cells imbedded in or adherent on carrier substances such as alginate, or other biocompatible substrates formed as beads or other small particulates. Cells may, for example, include any number of stem or proliferative cells including but not limited to mesenchymal cells, CD34+ antigen presenting cells, and neural progenitor cells.

While significant clinical research has addressed the activity of the cellular therapeutics, much less research has been devoted to the delivery of the cellular therapeutics to the target organs. Some work has progressed in delivering such substances to various organs such as the heart via a catheter system, or through the use of a small needle to inject directly into the myocardium.

While catheters or needles may be used as patient interface devices, the actual motive force to deliver the cells may arise from either manual injection, or from powering or automating the injection or infusion process. Powered devices having various degrees of automation may be based on a syringe delivery device or other means to power the flow of the cellular therapeutic containing fluid. While manual delivery is the most simple mode of delivery, it suffers from lack of fine control. Such parameters as flow rate, acceleration/deceleration rates, and other flow parameters are not easily controlled by hand. For instance, FIG. 1 shows the measured results of an actual hand injection of a fluid using a syringe compared to a powered or automated injection. The graph of FIG. 1 illustrates that a typical hand injection results in significant fluctuations in flow rate and acceleration rate as the user unsuccessfully attempts to deliver the fluid at a desired flow rate of 0.02 ml/s over a period of 90 seconds, while the powered or automated injection provides a relatively constant flow rate over the measure period of 90 seconds.

Parameters involved in injecting or infusing non-cellular fluids, such as solutions or suspensions of radio-opaque imaging contrast media, are generally chosen based on the procedure being performed, patient-based parameters and/or the ability to deliver the material to specific locations. For example, procedures aimed at resolving cardiac function using imaging contrast agents may use a complex profile of injection rates to assure a tight bolus of material entering the cardiac chambers or arteries. The general assumption for such procedures is that the injection rate will have no effect on the ability of the contrast material to attenuate the incident radiation during an imaging procedure.

However, cells and/or other biologics being living entities with complex structure and function or being derived from such living entities, may not be immune to the injection process or the environment created by the injection device. Studies suggest, for example, that stem cell behavior and response are affected by various physical forces. Factors which influence physical forces on cells, such as injection rate, injection acceleration/deceleration, length of tubing from a syringe, length, gauge or output hole configuration of a needle, etc. may all have an impact on the ability of the cells to survive or function according to their therapeutic design.

### SUMMARY

In a number of embodiments hereof, devices, systems, and methods are provided for improving the delivery of biologic, for example, cellular, therapeutics to a patient. For example, devices, systems, components and/or methods hereof may, for example, provide for auto-configuring or populating of determined (for example, partially or fully optimized) injection parameters for the injection or infusion of injectates including biologic-based, for example, cellular-based, therapeutic materials.

In one aspect, a system includes at least one pressurizing mechanism, a fluid path adapted to be placed in operative connection with the pressurizing mechanism to deliver an injectate to a patient, wherein the injectate includes cells, a control system operably associated with the at least one pressurizing mechanism; and at least one parameter generation system in operative connection with the control system. The parameter generation system includes an input system to receive data of a type (or types) of cells to be injected and is adapted to generate at least one parameter for the injection procedure at least in part on the basis of the data of the type of cells. The at least one parameter for the injection procedure may, for example, be a variable associated with the injectate, a variable associated with an injection protocol or a variable associated with at least one component of the fluid path.

The variable associated with the injection protocol may, for example, be a flow rate, an injection volume, an injection delay, duration of injection, an acceleration rate, or a deceleration injection rate. The variable associates with at least one component of the fluid path may, for example, be associated with a surface interaction of the cells and/or with the dynamics of flow through the component. The variable associated with at least one component of the fluid path may, for example, be an identity of the component, a volume, a length, an inner diameter, a material composition, a surface condition, an internal surface coating, or an internal geometrical configuration. In the case that the at least one component of the fluid path is a needle, the variable associated with the at least one component may, for example, be a length, an inner diameter, an outer diameter, a curvature, a material composition, a surface condition, an internal surface coating, an internal geometrical configuration, or a number of exit holes. In general, the fluid path includes any component or system through which the fluid including the cells passes to be delivered to the patient. As used herein, the term “fluid” refers to any flowable material or material capable of flowing. In a number of embodiments, such fluids or flowable materials may be, for example, mixtures, suspension or slurries of one or more liquids and insoluble matter. The variable associated with the injectate may, for example, be an initial concentration of cells, a temperature, an agitation or mixing condition, a composition or an injectate loading condition.

The parameter generation system may, for example, be adapted to be in communicative connection with a local memory system. The memory system may, for example, have stored parameters for injection procedures that may, for example, include parameters determined via at least one optimization study associated with each of a plurality of types of cells.

In a number of embodiments, the fluid path includes a container adapted to be placed in operative connection with the pressurizing mechanism so that injectate within the container can be pressurized for delivery. The container may, for example, be a syringe.

The at least one parameter may, for example, be chosen at least in part to control differentiation of a cell that has the ability to differentiate.

In another aspect, an injector system for use in connection with a fluid path to deliver an injectate to a patient wherein the injectate includes cells includes at least one pressurizing mechanism adapted to be placed in operative connection with the fluid path, a control system operably associated with the at least one pressurizing mechanism; and at least one parameter generation system in operative connection with the control system. The parameter generation system includes an input system to receive data of a type of cells to be injected and is adapted to generate at least one parameter for the injection procedure at least in part on the basis of the data of the type of cells.

In another aspect, a method of injecting an injectate including cells in an injection procedure includes placing a fluid path in operative connection with a pressurizing mechanism to deliver the injectate to a patient and generating at least one parameter for the injection procedure. Based on optimization studies, one cell characteristic may be optimized, or a plurality of cell characteristics may be optimized as group. Further, one parameter for the injection procedure may be optimized, or a plurality of parameters for the injection procedure may be optimized as a group.

In another aspect, a method of controlling the differentiation of a cell that has the ability to differentiate includes controlling at least one parameter of a fluid including the cell or of flow of the fluid including the cell through a fluid path.

In another aspect, a system includes at least one pressurizing mechanism, a fluid path adapted to be placed in operative connection with the pressurizing mechanism to deliver an injectate to a patient, wherein the injectate includes a biologic, a control system operably associated with the at least one pressurizing mechanism, and at least one parameter generation system in operative connection with the control system. The parameter generation system includes an input system to receive data of a type of biologic to be injected and is adapted to generate at least one parameter for the injection procedure at least in part on the basis of the data of the type of biologic.

In still a further aspect, a method of injecting an injectate including a biologic in an injection procedure includes placing a fluid path in operative connection with a pressurizing mechanism to deliver the injectate to a patient and generating at least one parameter for the injection procedure at least in part on the basis of the data of the type of biologic.

The present disclosure, along with the attributes and attendant advantages thereof, will best be appreciated and understood in view of the following detailed description taken in conjunction with the accompanying drawings.

### BRIEF DESCRIPTION OF THE DRAWINGS

FIG. 1 sets forth measured results of manual injection of a fluid using a syringe compared to injection of the fluid using a powered injector or automated injector.

FIG. 2 sets forth the results of injecting hCD34+ and mNSC cells using values for injection parameters at opposite ends of a suitability scale for each cell type.

FIG. 3A illustrates an embodiment of an injection system for delivery of cellular-based therapeutic materials.

FIG. 3B illustrates another embodiment of the injection system of FIG. 3A with an alternative fluid path.

FIG. 4 illustrates a system level block diagram of the injection system of FIG. 3A

FIG. 4A demonstrates the interaction between the methods described herein and the devices described herein.

FIG. 5 illustrates a flowchart of an embodiment of a method of using an automated injection system for delivery of cellular-based therapeutics.

FIG. 6 illustrates a flowchart of an embodiment of a method for programming the injector system database with determined injection protocol parameters and disposable component or system parameters to improve or optimize the delivery of a cellular therapeutic.

FIG. 7 illustrates an embodiment of a test matrix developed for mNSC characterization.

FIG. 8 sets forth antibodies used for mNSC differentiation assays.

FIGS. 9A and 9B set forth assay results of mNSC characterization tests.

FIG. 10 sets forth equations determined by experimental design analysis that relate injection parameters to various mNSC cellular responses.

FIG. 11 sets forth injector parameter values determined by non-linear simultaneous equation analysis to optimize tested mNSC cellular responses as a combined group.

FIG. 12 sets forth injection parameters values determined by non-linear simultaneous equation analysis to optimize only the proliferation of mNSC.

FIG. 13 sets forth injection parameters values determined by non-linear simultaneous equation analysis to minimize only the apoptosis of mNSC.

FIG. 14 sets forth injection parameter values determined by non-linear simultaneous equation analysis to optimize tested hCD34+ cellular responses as a combined group.

FIG. 15 sets forth injection parameter values determined by non-linear simultaneous equation analysis to optimize the differentiation of mNSC into astrocytes, oligodendrocytes, or neurons.

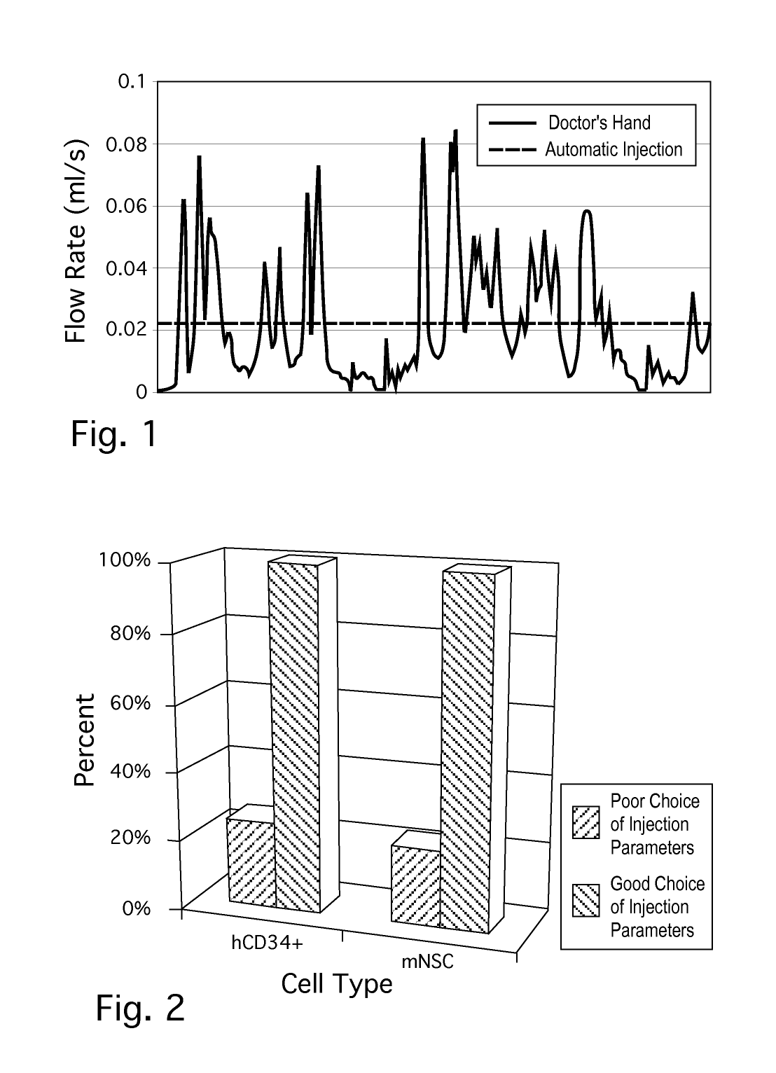

### DETAILED DESCRIPTION

As used herein and in the appended claims, the singular forms “a,” “an”, and “the” include plural references unless the context clearly dictates otherwise. Thus, for example, reference to “a type of cell” includes a plurality of such types of cells and equivalents thereof known to those skilled in the art, and so forth, and reference to “the type of cell” is a reference to one or more such type of cells and equivalents thereof known to those skilled in the art, and so forth.

Although it is known that physical forces and/or stimuli can negatively affect cells during an injection procedure, the present inventors have discovered that there are significant differences between the reactions of different types of cells and other biologic materials or biologics to, for example, physical stimuli during an injection procedure. As used herein, the terms “biologic materials” or “biologics” refer to therapeutic materials that are created by biologic processes, rather than being chemically synthesized. Biologics may, for example, be isolated from a variety of natural sources including human sources, animal sources, and/or microorganism sources. Certain biologics may be produced by biotechnological and/or other methods. Biologics include, for example, cell therapies, gene therapies, viral therapies etc. Representative examples of devices, systems and methods hereof are discussed in connection with representative examples of injectates including cells. However, the devices, systems and methods hereof are applicable to, for example, the handling, injection and/or delivery of injectates including other biologics.

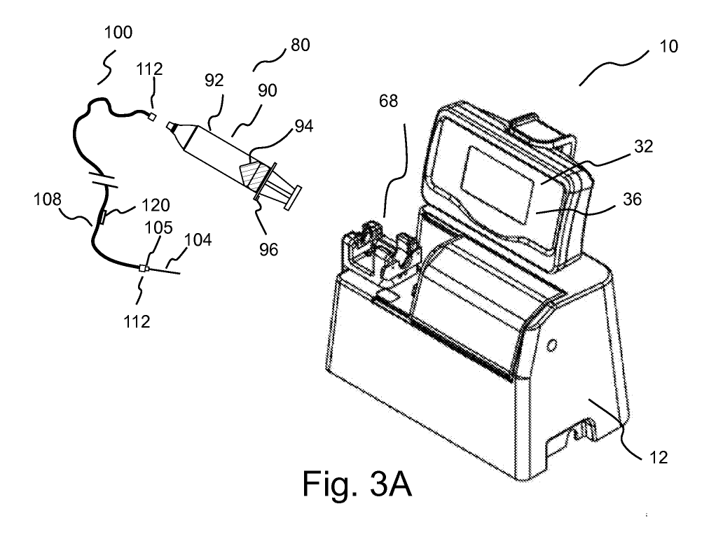

For example, representative studies using human bone marrow (hCD34+) cells, mouse embryonic neurospheres (mNSC), and mouse stromal cells indicate that injection parameters determined, designed or optimized for one cell type may not be equally optimal for another cell type. FIG. 2 illustrates the results of representative studies including injection of hCD34+ and mNSC cells using values for injection parameters at opposite ends of a suitability scale. If injection parameter values are chosen well for a particular cell type, as many as 100% of the cells can be dispensed through the delivery system to the target. If injection parameters are chosen poorly for a particular cell type, almost 80% of the cells will not be delivered. Similar relationships were obtained for other measured cell assays. Moreover, it was found that by choosing the appropriate injection parameters, cells capable of differentiating could be directed to more (or less) readily differentiate into specific cell types.

In a clinical setting, it is not reasonable to expect that a physician or technician operating a device or system to deliver cells would be aware of parameter settings for the system (including, for example, the use of cell-specific disposable fluid path components such as a syringe, tubing, a catheter, or a needle) for any of a plurality of cellular therapeutic materials. It is thus desirable to provide a system to generate, determine and/or auto-configure injection parameters for a given a cellular therapy material. An injection system or device may, for example, auto-configure or auto-populate parameters for an injection procedure using a parameter generation system which generates one or more parameters for the injection procedure at least in part on the basis of the data of the type of cell(s) to be delivered.

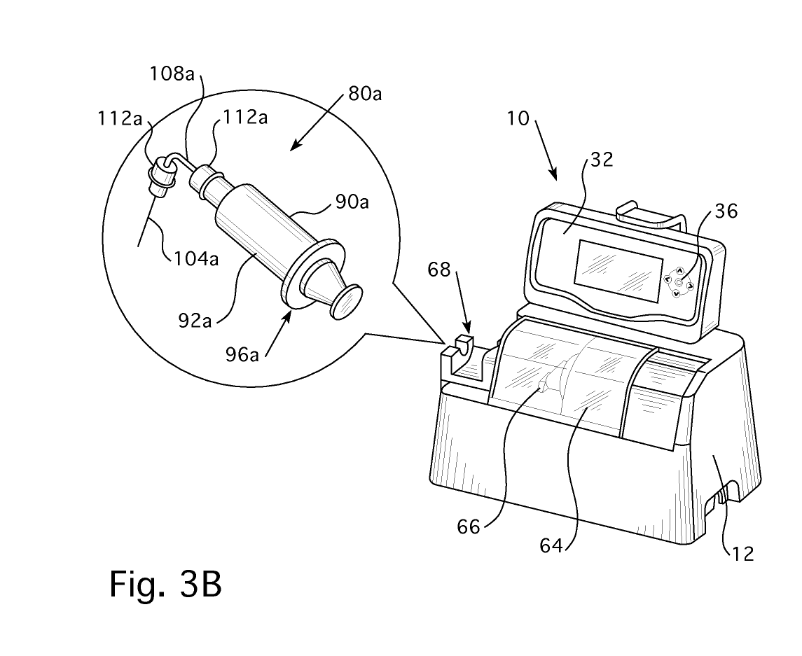

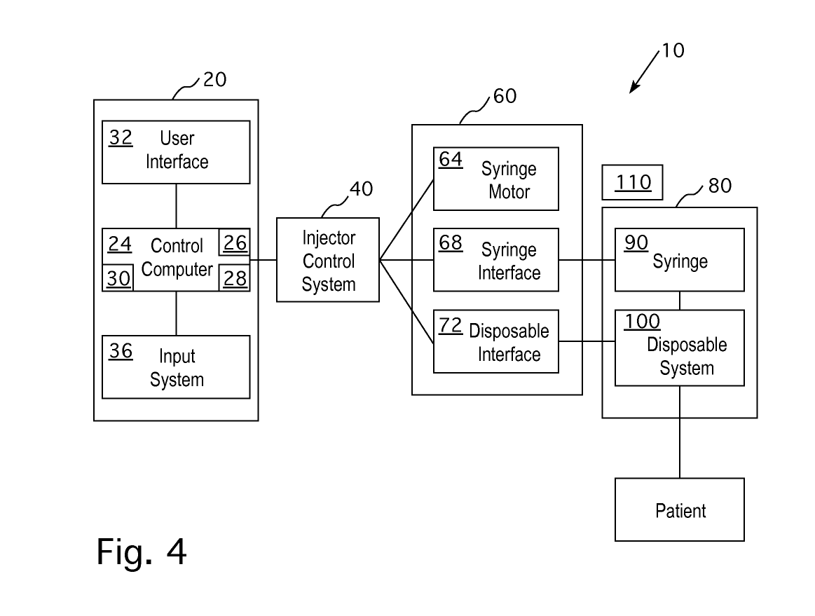

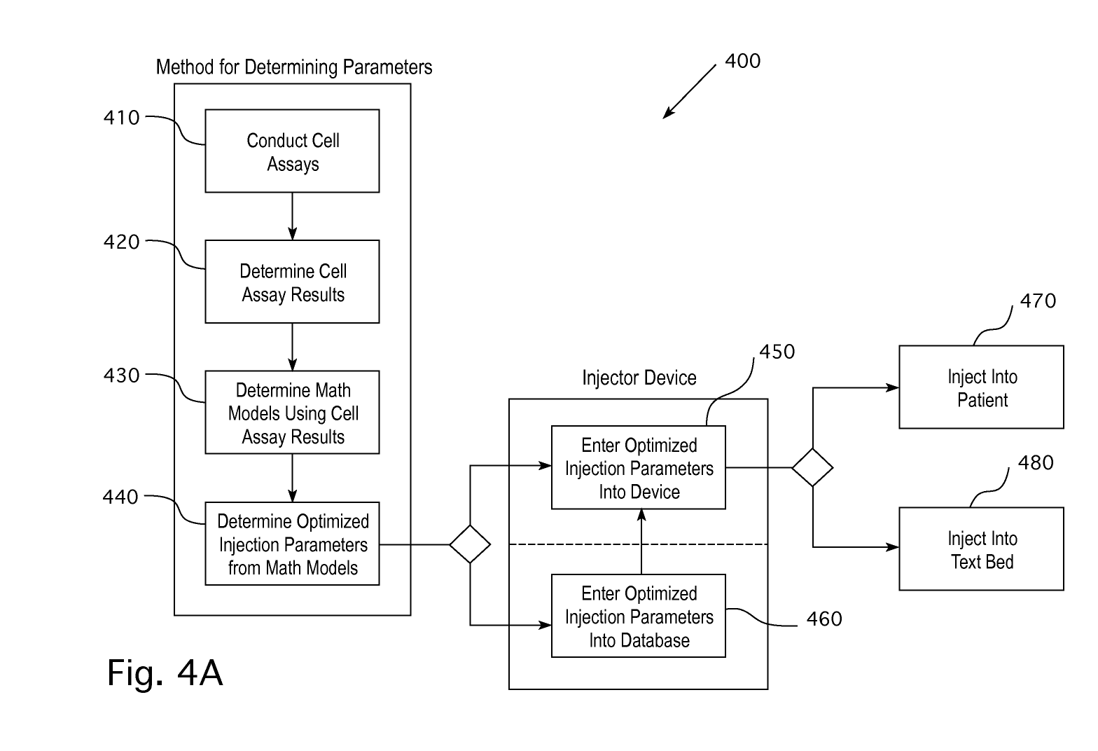

FIG. 3A illustrates an embodiment of an injection system, delivery system or system 10 for use in delivering an injectate including cells. Powered or automated injection systems hereof may, for example, be stationary or handheld. FIG. 4 illustrates a system level diagram of injector system 10. In the illustrated embodiment, system 10 includes a computer system or subsystem 20, an injector control system, subsystem or controller 40, an injector system or subsystem 60, and a fluid path system or subsystem 80 including, for example, a syringe 90 and a disposable system 100 which may include one or more disposable fluid path components. Syringe 90 may also, for example, be disposable. In system 10, computer system 20, injector control system 40 and injector system 60 are positioned at least partially within a housing 12.

Fluid path elements hereof may, for example, be disposable after a certain period of use, upon a per-patient basis or upon a per-procedure basis. Injector systems and fluid path components suitable for or adaptable for use herein are also described, for example, in U.S. Patent Application Publication Nos. 2007/0106208, 2011/0028908 and 2008/0294096, the disclosures of which are incorporated herein by reference. Although representative examples of system 10 are described for use with syringe 90, other pressurizing systems and mechanisms and other containers (for example, collapsible containers, bottles, vials etc.) may, for example, be used in the systems hereof.

FIG. 3B illustrates system 10 with an alternative fluid path system 80a. Elements of fluid path system 80a are number similarly to corresponding or like elements of fluid path system 80, with the designation “a” added thereto. A portion of housing 12 is illustrated as transparent in FIG. 3B to illustrate elements of injector system 60.

In the illustrated embodiment, computer subsystem 20 includes a control computer or controller 24 which may, for example, include one or more microprocessor boards 26, one or more memory systems 28, a user interface system 32 (including, for example, one or more computer monitors or screens), and an input system 36 (including, for example, a keyboard, mouse, one or more sensors, and/or other electronic input devices). In the embodiments of FIGS. 3A and 3B, user interface 32 and input system 36 are embodied, at least partially, in a touch screen. Computer subsystem 20 may, for example, take input from the user and/or from one or more sensors regarding the nature of the cellular therapeutic material either by cell type, trade name, or other indicator. The user may also input other information regarding the patient or the protocol under which the therapeutic may be delivered to the patient. Control computer 24 may, for example, provide information to the user regarding the injection procedure including but not limited to recommended injection parameters and doses for the therapeutic, or a description of a preferred syringe or disposable system that is determined or predetermined for the use of the therapeutic material. Other information regarding the injection procedure may, for example, be provided to the user by injector subsystem 60 via a system output device or via user interface 32, such as a verification message that the determined (for example, determined to be suitable, recommended or optimized) syringe or determined disposable component for a therapy including delivery of a specific cell type has been properly associated with injector subsystem 60, or that an unknown or non-determined syringe or disposable component has been associated with injector subsystem 60. Control computer 24 may further contain or be in communicative connection with a store of information or data, stored, for example, in a database that may, for example, configure the operation of a syringe motor 64 according to the type of therapeutic used in the injection procedure. Such a set of data, variables, parameters or instructions for operation of syringe motor 64 is sometimes referred to herein as an “injection protocol” or a “protocol.” Such information may include, but is not limited, to the average injection rate, the acceleration and deceleration profile for the injection, amount of material to inject at any one time, number of individual injections of the therapeutic, delay times between sequential injection, and similar information. Various parameters for injection protocols are, for example, discussed in U.S. Patent Application Publication Nos. 2008/0097197, 2007/0282263, 2010/0113887 and WO 2009/012023, the disclosures of which are herein incorporated by reference for this purpose. Further, control computer 24 may also include and/or be in communicative connection with a store of information regarding a type of syringe 90 or a type of one or more components of disposable system 100 that is determined for the use of the particular therapeutic. The information in the database may, for example, be updated or edited by a programmer or injector user by causing injector subsystem 60 and/or injector system 10 to enter into a programming mode for this purpose. This database may be stored locally in memory system 28 of control computer 24.

Computer subsystem 20 is in electrical and communicative connection with injector control system or controller 40. Computer subsystem 20 and injector control system 40 may, for example, be partially or fully integrated into a single computer system or may be distributed over more than one computer system. Injector control system 40 provides control data to syringe motor 64 to activate motor 64 according to the protocol defined and/or determined (for example, via one or more optimization procedures) for the type of therapeutic material to be used in a specific injection procedure. Injector control system 40 may also receive information from syringe motor 64 and/or other devices or systems. In a representative example, such information may, for example, include data from a syringe plunger velocity sensor (represented generally by sensor system 110) that monitors whether syringe 90 is operating according to the injection protocol. Injector control system 40 may, for example, also receive information from a sensor (represented generally by sensor system 110) associated with a syringe interface 68, to determine/indicate whether a syringe 90 determined for the cellular therapeutic has been associated with syringe motor 64 via syringe interface 68. Further, injector control system 40 may, for example, receive information from one or more sensors (represented generally by sensor system 110) associated with a disposable interface 72 to determine/indicate, for example, whether a disposable fluid path system or component determined for the cellular therapeutic has been associated with syringe 90. The information received from these components or sensors may, for example, be returned to control computer 24 for display to the injector system user (via, for example, user interface 32) and may further be used by control computer 24 to prevent injection if the parameters and/or fluid path components are not those determined for the injection of the cellular therapeutic.

Injector subsystem 60 provides motive force to syringe 90 to inject or infuse an injectate including a cellular therapeutic into the patient. As described above, injector subsystem 60 may, for example, include at least one syringe motor 64, at least one syringe interface 68, and at least one disposable interface 72. As known in the art, syringe motor 64 is adapted to be placed in mechanical communication with syringe 90 (or other container), and operates on syringe 90 (or other container) to drive or cause motion of a cellular therapeutic therein. Such motions may include, but are not limited to, aspirating the cellular therapeutic from a source container, injecting the therapeutic into disposable system 100, which is in fluid communication with syringe 90, and agitating the cellular therapeutic in syringe 90 to, for example, keep the cellular therapeutic in fluid suspension. In other representative embodiments (not shown), injector subsystem 60 may also include a separate motor or other motive device capable of agitating the cellular therapeutic in syringe 90 to, for example, maintain the cellular therapeutic in fluid suspension. In a representative embodiment, such a motive device may, for example, include a piezoelectric stack in physical communication with syringe 90 which, when activated, vibrates and/or translates syringe 90 and its contents. Syringe motor 64 may also include one or more sensors (represented generally by sensor system 110) to determine that syringe motor 64 is operating according to the protocol communicated to it from control computer 24 via injector control system 40. In a representative embodiment, a syringe motor sensor may, for example, measure current energizing syringe motor 64 to determine a flow rate.

As described above, injector subsystem 60 includes syringe interface system 68, which may, for example, be adapted to interface with, at least partially house and/or stabilize syringe 90. As known in the injector arts, injector subsystem 60 may also include a coupling 66 to form an operative engagement between a syringe plunger 94 (see, for example, FIG. 3A) and syringe motor 64, a stabilizing component to assure a syringe barrel 90 remains in a determined position, and one or more sensors (represented generally by sensor system 110) to read identification information from indicia 96 on syringe 90 that may be communicated to control computer 24 via injector control system 40. In a number of embodiments, syringe interface system 68 may also include a coupling device from syringe 90 to a motive source (not shown) to agitate the cellular therapeutic, thereby assuring that the cellular therapeutic is, for example, maintained as a reasonably homogenous suspension. In a number of embodiments, syringe interface system 68 may also include a temperature control system (for example, including a shield or cover (not shown)) that may, for example, be adapted to maintain the cellular therapeutic within a determined temperature range.

As also described above, injector subsystem 60 also includes disposable interface system 72 which is, for example, adapted to interface with, at least partially house and/or stabilize disposable system 100 which includes one or more disposable fluid path components. Further, disposable interface system 72 may include one or more sensors (represented generally by sensor system 110) to read identification information from indicia 120 (see FIG. 3A) of a disposable system 100 or from indicia of one or more disposable fluid path components of disposable system 100 (for example, indicia 105 of needle 104, as illustrated in FIG. 3A) that may, for example, be communicated to control computer 24 via injector control system 40.

Syringe 90 of injection system 10 contains the cellular therapeutic for delivery to a patient. As known in the art, syringe may include barrel 92, plunger 94 (which is reciprocally slidable within barrel 92), along with any of a plurality of seals, flanges, or information/data indicia 96. Indicia 96 may, for example, include user readable information to identify syringe 90 and/or its contents, and markings to indicate fluid volume. Syringe 90 may, for example, be constructed of a variety of materials including, but not limited to, glass, or plastics such as polycarbonate, polyethylene or polypropylene. Further, syringe 90 may be coated on the interior surface thereof with material specifically designed for compatibility with the cellular therapeutic. Such coatings may, for example, include silicon oils, hydrophilic materials or nanotechnology coatings. In a number of embodiments, indicia 96 of syringe 90 includes a machine-readable component to provide information related to the identity of syringe 90 and/or its contents. Such a machine-readable component may, for example, include a bar-code, a magnetic strip, radiofrequency identification (RFID) device, or one or more identifying physical features molded into syringe 90 such as detents, flanges, or protrusions. Syringe indicia 96 may, for example, be detected by a sensor (represented generally by sensor system 110) incorporated in syringe interface system 68, such as, for example, a bar code reader or an RFID reader. The data encoded in syringe indicia 96 may, for example, be sensed by the syringe interface sensor, which, in turn, may relay the information to control computer 24 via injector control system 32.

Disposable system 100 is in fluid communication with syringe 90, and transfers the injectate including the cellular therapeutic from syringe 90 into the patient. Disposable system 100 may, for example, include any number of transfer or fluid path components, including but not limited to one or more needles or catheters 104, tubing 108, connectors 112, and/or combinations thereof (see, for example, FIG. 3A). Needles 104 may, for example, be fabricated from metal or plastic, and may have an internal coating such as , but not limited to, a silicon oil or a nanocoating. Further, needle 104 may have a single or multiple outlet orifices through which the therapeutic may flow into the patient. The orifices may be disposed at the end of needle 104, about the shaft of needle 104, or according to any other geometric distribution. The end of needle 104 may be tapered in a beveled edge or blunt, and the shaft of needle 104 may be straight or curved. Tubing or a catheter used as a disposable may be fabricated from any number of materials including, but not limited to, silicon rubber, or other plastic such as nylon or polytetrafluoroethylene (PTFE).

As described above, disposable system 100 may include indicia 120, which may be machine-readable, to provide information related to the identity of disposable system 100 and/or components thereof. Indicia 120 may include, but not be limited to, a bar-code, a magnetic strip, a radiofrequency identification device, or identifying physical features molded into disposable system 100 such as detents, flanges, or protrusions. Indicia 120 (and/or indicia of individual components of disposable system 100 such as indicia 105 of needle 104) may be detected by a sensor (represented generally by sensor system 110) incorporated in the disposable interface. The data encoded in the syringe indicia 96 and disposable system indicia 120 may be sensed by the one or more sensors which, in turn, may relay the information to control computer 24 via injector control system 40.

Fluid path system 80 (including, syringe 90 and disposable system 100) may, for example, be determined (using, for example, optimization methods as described further below) and made available to a user of system 10 as an integral unit. Alternatively, syringe 90 may be determined and made available separately from disposable system 100. Further, one or more components of disposable system 100 may be determined and made available separately.

In operation of a number of embodiments hereof, control computer 24 configures injector control system 40 with injector parameters after the user and/or one or more sensors of sensor system 110 has identified to system 10 the type of cellular therapeutic or injectate that is being injected. The data regarding determined injection procedure parameters may be transferred from the system database or another connected database to, for example, injector control system 40 for this purpose. Further, control computer 24 may respond in a variety of ways when non-determined (that is, not determined for use with a specific cell type or cellular therapeutic using, for example, an optimization method as described below) or unrecognized syringe(s) and/or disposable components are associated with injector subsystem 60. In a number of representative examples, system 10 or injector subsystem 60 may default to a non-functional state in response to non-determined components and/or issue a warning to the user, injector subsystem 60 may default to a standard or default set of injector parameters and issue a warning to the user that the default parameters will be employed, or injector subsystem 60 may only provide a warning to the user and execute the programmed parameters.

In a number of embodiments (not shown), injector system 10 may include multiple syringes, each associated with a separate disposable system, or with a disposable component capable of mixing the contents of the multiple syringes. The multiple syringes may be used to hold multiple contents such as, for example, a cellular therapeutic, growth enhancing or stimulating cytokines, chemicals capable of retarding apoptosis or stimulating differentiation, diluents etc. Those contents may, for example, be mixed by a mixing disposable immediately before injecting the material. Alternatively, the contents may be injected sequentially. Each syringe may, for example, be associated with a separate syringe motor and interface, each capable of acting independently.

FIG. 4A shows a high level interaction, 400, between the methods described herein and the devices described herein. One method includes actions to conduct cell assays on specific cell types, 410, as otherwise described. Once the results of these assays are determined, 420, they are used to develop math models identifying the relationships between injection parameters and cell function, 430. These math models are then used to determine the optimum values for each cell function using appropriate statistical analyses, 440. These values can then either be manually entered into the injector device, 450, or entered into a database that communicates these values to the injector device, 460. The injector uses these values to configure itself to inject the cells into either a patient, 470, or alternatively into a test bed, 480, for example, for use in drug development, drug discovery, or other research or production activities.

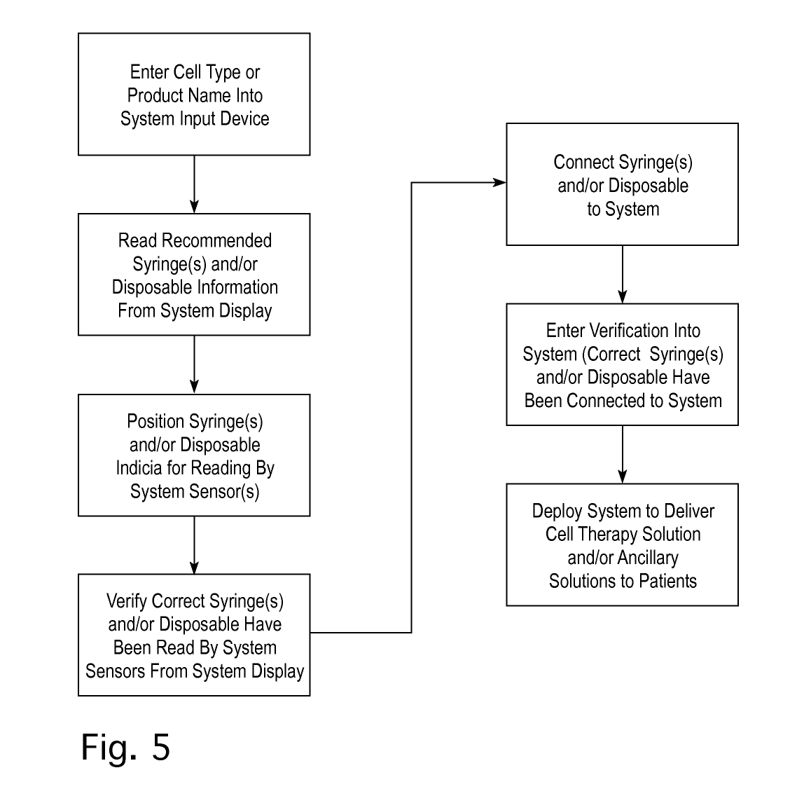

FIG. 5 illustrates an embodiment of a method whereby a parameter generation system hereof configures variables or parameters for an injection procedure to deliver an injectate including cellular-based therapeutics to a human patient. As described above, injector system 10 includes or is in communication with a database including data related to injector parameters determined for a specific type of cellular therapeutic (that is, a specific type of cells or combination of cells). The parameter generation system, which may, for example, be embodied (at least in part) within software saved in, for example, memory 28 of computer system 24 and/or another computer or control system, is adapted to generate at least one parameter for the injection procedure. The parameter may, for example, be a variable associated with the injectate, a variable associated with an injection protocol or a variable associated with at least one component of the fluid path. Such parameters include, but are not limited to, the rate of fluid injection, total fluid volume for any single injection step, the acceleration and/or deceleration rates for the fluid injection, a lag time between successive injection steps, the name, type, or other identifying information related to a container or containers (for example, a syringe or multiple syringes) containing the cellular therapeutic material, the name, type, or other identifying information related to disposable fluid path components such as tubes, cannulae, connectors, needles, and the nature of any other material to be injected with or after the cellular therapeutic material.

In the embodiment of the method illustrated in FIG. 5, data of a cell type is first entered into injector system 10 via, for example, input system 36, for example, via manual entry by an injector user or via sensor system 110. A physician or technologist may, for example, enter cell or product identifying information into injector system 10 via an input device of input system 36 such as a keyboard. Such information may also be entered by a user via selection from a drop-down menu displayed, for example, by user interface 32. Such information may additionally or alternatively be entered (and/or confirmed) via a sensor which reads information from, for example, a container containing the cellular therapeutic material. The information entered into injector system 10 may, for example, be communicated to the parameter database. The determined parameters listed in the parameter database associated with the identified cell or cellular therapeutic material may, for example, then be displayed to the user via user interface 32.

As illustrated in FIG. 5, the user may read the determined parameters displayed by user interface 32. Such information may, for example, include an identification or recommendation of fluid path system or components thereof such as syringes and/or components of disposable system 100 that have been determined (for example, optimized) for the delivery of the identified cellular therapeutic. In a number of embodiments, injector system 10 uses determined injector protocol parameters (such as, for example, rate of injection, etc.) and programs injector control system 40 to apply the determined parameters to the injection protocol for the specific injection procedure. Injector system 10 may, for example, display the injector protocol parameters (such as, for example, injection rate, acceleration/deceleration rates, etc.) to the user who then actively programs injector control system 40 with those injection protocol parameters. Along with the injector protocol parameters, injector system 10 may provide the user with information regarding the determined syringe(s) 90 and/or one or more determined components of disposable system 100 (for example, needle 104) determined (for example, optimized) for use with the cellular therapeutic input into injector system 10. In a number of representative examples, such information may, for example, include written descriptions of the components (such as a 10 ml syringe by a particular manufacturer, and/or a 1 inch, 22 gauge needle with a beveled edge), serial numbers identifying products produced by the system manufacturer specifically designed for use with the system, or other identifying information (such as package color) that a user will recognize as identifying particular components for use with injector system 10.

Based on the identifications or recommendation provided by the parameter generation system of injector system 10, a user may, for example, obtain the syringe(s) and/or disposable system components for use with injector system 10. Once again, such fluid path components may, for example, have indicia associated therewith that may be sensed by one or more sensors of injector system 10. The user may, for example, place the syringe(s) and/or other disposable system components in proximity to associated indicia sensors so that injector system 10 may determine that the fluid path components identified or recommended by the parameter generation system have been chosen by the user.

Injector system 10 may then present information via user interface 32 to indicate to the user that the indicia have been read to confirm that the syringe(s) and/or disposable system components have been identified by injector system 10 as correct. The user may, for example, use this information to verify that injector system 10 has recognized the fluid path components.

Thereafter, the user may connect the syringe(s) and/or disposable components to the injector system. Upon connecting these components, the user may then notify the system via an input device such as a keyboard that the components have been connected. In an alternative embodiment, various identifying indicia may be used by the injector to verify that the components have been correctly connected. For example, a syringe possessing a specific configuration of flanges may be recognized by an appropriate sensor associated with syringe interface 68. The output of the sensor may be relayed to injector control system 40 to verify the placement of the syringe.

Once the syringe(s) and/or disposable components have been connected to injector system 10, and the connection has been verified by the system, the user may then deploy injector system 10 for delivering the cells. As described above, in the event that the syringe(s) and/or disposable components are not those recognized by injector system 10 as being determined/optimal for the cellular therapeutic material, the system may be disabled from delivering the therapeutic material, the system may issue a warning to the user that the sub-optimal components have been delivered, or the injector system may operate in a default mode as specified in the injector design.

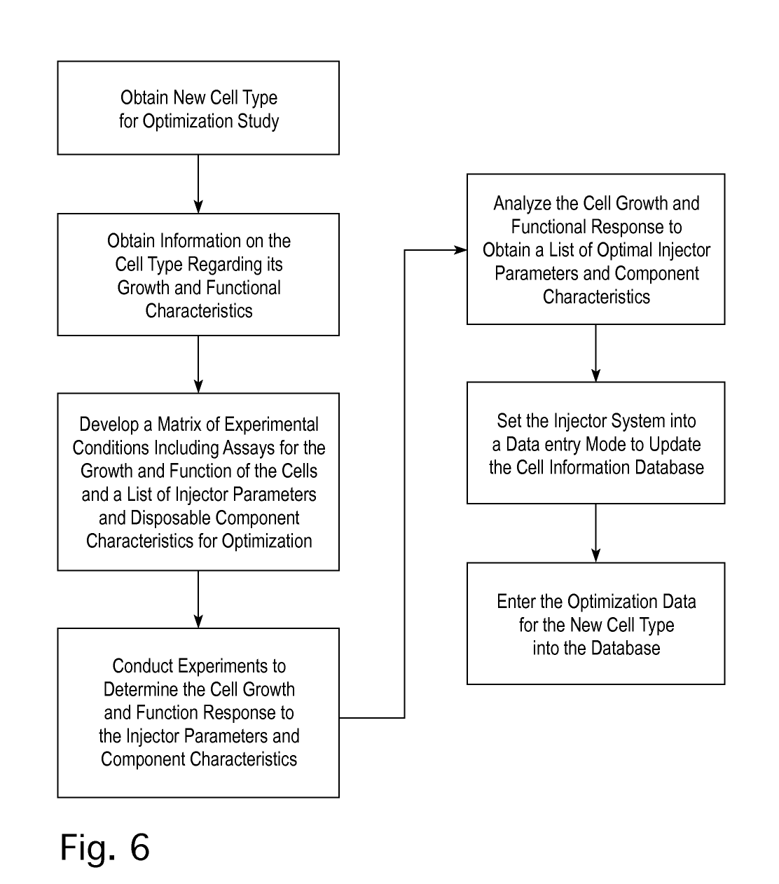

FIG. 6 illustrates an embodiment of a method wherein a cellular therapeutic device with, for example, an internal database including determined data regarding parameters for injection procedures for a number of cell types may be programmed to include new data for a specific cell type or cellular therapeutic not already included in the database. In the embodiment of FIG. 6, the programmer of the database first obtains the new cellular therapeutic, or the cell type(s) within the cellular therapeutic, from a source. The programmer may, for example, physically come into possession of the cells or make arrangements for a third party to obtain this material on his behalf.

The programmer also obtains relevant information regarding the therapeutic material. This information may, for example, include means of culturing and/or handling the therapeutic material (for example, the appropriate growth medium in which to maintain the cellular therapeutic or the cells it contains), means of expanding the number of cells if necessary (for example, specific growth conditions such as feeder cell layer, temperature, and other laboratory conditions), and information related to the specific therapeutic function of the therapeutic (such as elaboration of specific metabolytes, growth factors, or the function of specific differentiated progeny from the therapeutic).

In the embodiment of FIG. 6, a test matrix of therapeutic assays and injection system parameters is developed. The therapeutic assays may, for example, be related to the cell growth, survival, identity, and function. Several representative examples of each of these assays follow. Cell growth may, for example, be assayed through tritiated thymidine uptake. Cell survival may, for example, be assayed through trypan blue staining for viability, total cell counting, and testing for apoptosis (such as caspase activation). Cell identity may, for example, be assayed through the identification of cell surface markers through antibody staining, morphology, and the ability to grow under specified growth conditions, through reference to known nucleic acid sequences. Functional assays may, for example, include the ability of stem cells to differentiate into known progeny (such as neural stem cells being able to differentiate into astrocytes, neurons and oligodendrocytes) which may, for example, be assayed by morphology, cell surface markers, their ability to elaborate specific metabolytes or proteins, or specific determination of up- or down-regulated genes through their resulting gene products.

Injection protocol parameters for study may include, but are not limited to, the rate of fluid injection, the acceleration or deceleration rates of the injection, the number or concentration of cells in the initial injectate, and the delivery volume of any one injection cycle. Characteristics of any syringe component may include without limitation the volume capacity, internal diameter, syringe material (such as glass or plastic), any required internal syringe coating (such as silicon oil, or hydrophilic coating), or the geometry of an exit feature (such as tapered or un-tapered, with or without Luer locks) either on the external surface or internal surface of the syringe. Characteristics of the disposable components may include without limitation the length and diameter of any delivery tubing or cannula, the material composing such tubing, the length, gauge, curvature, or material of any needle including the disposition of effluent portals (at the tip or along the needle shaft) and the shape of the needle tip (beveled, squared-off, or rounded). The characteristics of the disposable components may also include the type of interconnecting features or connectors between the disposables or between disposables and any syringe including any external or internal geometric factors such as a tapered or curved fluid path from one component to the next.

As described above, one or more entire fluid path systems such as fluid path system 80 and/or 80a may be determined for use in connection with a specific cellular-based therapeutic. Such fluid path system may, for example, be provided with associated identification numbers, names, indicia etc., and may, for example, be distributed as a system in sterile packaging unit. Individual components of a fluid path system may, for example, be made available in a similar manner.

For the test matrix, multiple values for the injection system parameters may, for example, be specified and the therapeutic assays run for the injection system configured to the various values. The number of parameter values for continuously varying parameters may, for example, be varied to include the endpoints of a range of possible values, a linear list of parameter values, or a more complex list of values (for example on a logarithmic range). For non-continuously varying parameters (such as disposable material type), a parameter value may represent each of a list of possible parameter values such as glass syringe, TEFLON® (a fluorine-containing, polymeric material (for example, polytetrafluoroethylene) available from E.I. Dupont de Nemours and Company of Wilmington, Del.) tubing, and similar non-countable values. The test matrix may specify a single parameter change from a baseline configuration as one test case, a subset of multiple changes from the baseline configuration as test cases (as used in a fractional design of factorial experiment), or all possible combinations of parameter changes as a number of test cases (such as a full factorial analysis).

After the experimental design has been developed, the programmer may, for example, run the experiments or arrange for a third party to carry out the experiments to provide the data. The data present the results of the cellular therapeutic assays when the cellular therapeutic material is injected according to the various injector system configurations specified by the parameter values in the test matrix. The programmer or a specified other party may then analyze the data of the experiment according to various known analysis methods to determine the optimal injector system characteristics and parameters. Such analyses may, for example, include an ANOVA (or analysis of variance) statistical model, a linear regression analysis, a non-linear parametric analysis, a response optimization analysis, or other statistical analysis known to those experienced in the art. Such analyses may also include without limitation the consideration of only one cell assay result, or combinations of cell assay results.

Once the optimized information has been determined, the programmer may cause the injector system to enter a programming phase in which the database may be updated with the new information. For example, the equation and coefficients determined by linear regression analysis could be entered relating important factors to, for example, delivery of a cell type such as human hematopoietic stem cells or hCD34+. In addition, optimization analysis may, for example, be performed to identify the values for each of the various factors that would allow the greatest number of cells to be injected for the conditions being tested. This information may also be entered into the database. Such a process may be followed for any other cellular assay that has been performed on the cell type without limitation. The programmer may also determine if any currently used syringe and/or disposable components that are determined/indicated as optimal for previously programmed cell therapeutics, may be optimal for the new therapeutic material. If so, the database may be updated with references to those components. If a new component or syringe is required, the programmer may provide or have a third party provide such a component with, for example, added indicia for component identification by the injector system.

A representative example of acquiring cell characterization data as set forth in the above-described method is set forth below in collecting data and identifying relationships between various operational and dimensional injection parameter for mNSC functionality.

Using experimental design methodology, a test plan was created to determine the effect of various operational and dimensional injection factors on mNSC survivability during an injection procedure. The methodology included a ½ fractional factorial design for 2-level experiments with 3 replicates, for a total of 48 individual tests (or runs). Such an experimental design is termed a Resolution V design, meaning that no main effect or two-factor interaction is aliased (or confounded) with any other main effect or two-factor interaction, but two-factor interactions are aliased with three-factor interactions. This means that the estimate of the interaction effect of each main factor is separate from the other main factors. Five factors at 2-levels were chosen to be tested:

1. Flow rate (2 levels: slow (0.1 ml/s) and fast (1 ml/s));

2. Needle Gauge (2 levels: small (27 gauge) and large (20 gauge));

3. Needle Length (2 levels: short (1.5″) and long (6″));

4. Initial Cell Concentration (2 levels: low (1.5×10⁶ cells/ml) and high (4×10⁶ cells/ml)); and

5. Acceleration/Deceleration Rate (2 levels: low (2.75 ml/s²) and high (19.27 ml/s²)).

Seven cell responses to these factors were measured:

1. Fraction of total mNSC after injection;

2. Fraction of viable mNSC after injection;

3. mNSC that proliferated (normalized) after injection;

4. mNSC that died (normalized) after injection (apoptosis);

5. mNSC that differentiated after injection into astrocytes (GFAP normalized);

6. mNSC that differentiated after injection into oligodendrocytes (O4 normalized); and

7. mNSC that differentiated after injection into neurons (β-tubulin normalized).

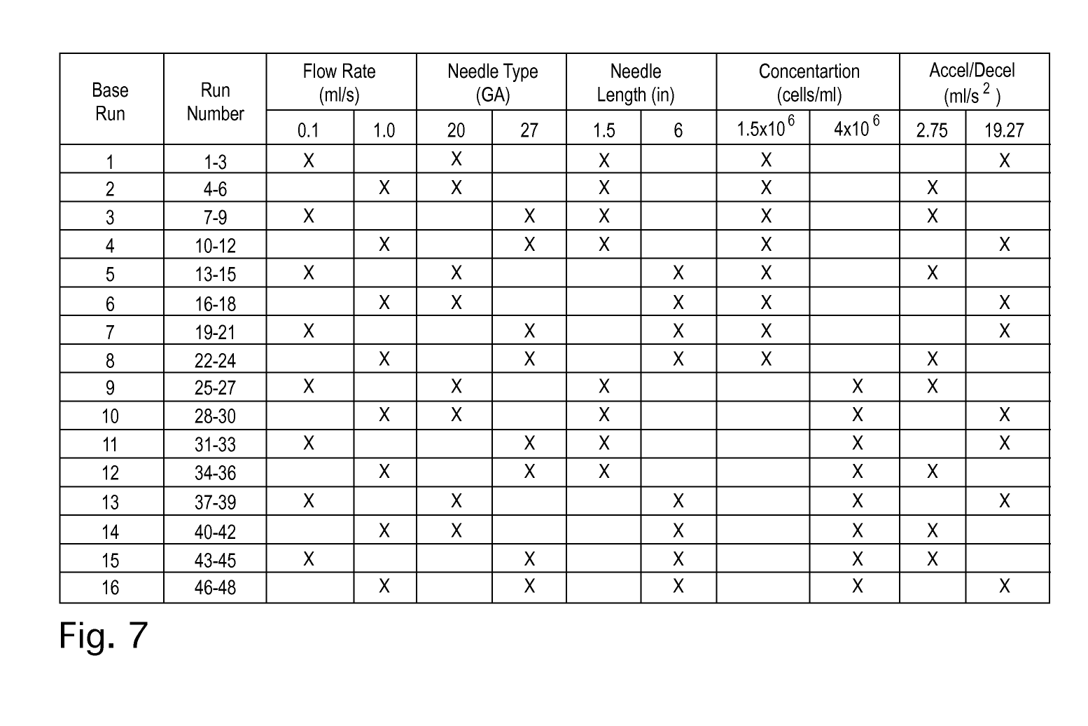

The cellular responses included those to assess cell survivability (final total cell number, cell viability, and apoptosis assessment)) as well as phenotypic stability (proliferation potential and differentiation assessment). The test matrix developed for the ½ fractional factorial design showing the specifications for each test is shown in FIG. 7.

Cryopreserved embryonic 14-day neurospheres derived from the ventral area of the mouse brain were obtained from a cell supplier (StemCell Technologies, Vancouver, Canada). Neurocult NSC proliferation medium (StemCell Technologies, Vancouver, Canada) was then warmed to 37° C., and 9 ml of it was added to a sterile centrifuge tube. A cryovial containing the frozen mouse neurospheres was then thawed quickly in a 37° C. water bath and 1 ml of the proliferation media was added drop-wise to the cryovial. The contents of the cryovial were then transferred to a centrifuge tube containing 9 ml of the proliferation media and centrifuged at 400 RPM for 5 minutes. Following centrifugation, media was aspirated from the cells and the neurospheres were resuspended in 10 ml of fresh proliferation media by gentle pipetting. The resulting single cell suspension was transferred to an appropriate cell culture flask containing a final volume of 30 ml of proliferation media and placed in a 37° C., 5% CO2 humidified cell culture incubator to increase the number of cells for eventual testing. The culture was checked daily to ensure that the floating neurospheres were intact and showed signs of expansion. Once the neurospheres reached approximately 100 μm in diameter (approximately 7 days), all the neurospheres were removed from the flask and centrifuged at 400 RPM for 5 minutes. Following centrifugation, the majority of the medium was aspirated from the tube leaving approximately 50 μL, to which 1 ml of TrypLE Express (Invitrogen Corporation, Carlsbad, Calif.) was added to facilitate cell dissociation; the tube was then placed in a 37° C. water bath for approximately 20 minutes. Cells were then centrifuged at 800 RPM for 5 minutes, the TrypLE Express aspirated from the tube, and 1 ml of NeuroCult NSC basal media (StemCell Technologies, Vancouver, Canada) was added. Basal medium contains the basic common nutrients the cells need to survive, but no signaling molecules/growth factors to drive the cells to proliferate or differentiate. The neurospheres were then resuspended by gently pipetting up and down (approximately 70-80 times), to form a single cell suspension and 9 ml of fresh basal media was added. The cells were then counted for a baseline using a Vi-CELL XR Cell Viability Analyzer (Beckman Coulter, Fullerton, Calif.). Using the techniques described, one vial of cryopreserved neurospheres containing approximately 5 million cells yielded approximately 60 million cells following 10 days of culture.

For actual injections, neurospheres were prepared as a single cell suspension in basal medium as described above and analyzed for cell concentration and viability. The cells were then adjusted to either 1.5×10⁶ cells/ml or 4×10⁶ cells/ml in the basal medium (as appropriate for a given test), loaded into a 5 ml syringe attached to the cell therapy injection device, and injected using the appropriate injection parameters into a vessel containing an appropriate volume of basal media to adjust the cell concentration to 1×10⁶ cells per ml. The injection procedure was performed within approximately 10 minutes of cell preparation. Following injection, the cells were again analyzed for cell concentration and viability, and the desired number of cells seeded into appropriate 96-well plates for the functional assays. The plates for the functional assays contained 2× proliferation media (for proliferation and apoptosis assays) or 2X differentiation media (for differentiation assay) as appropriate. The cells were added to the plates for the corresponding functional assay within approximately 10-15 minutes of preparing the single cell suspension. Functional assays were conducted as described below.

For the cell concentration and viability assays, 1 ml of single cell suspension was analyzed for cell concentration and viability using a Vi-CELL XR Cell Viability Analyzer (Beckman Coulter, Fullerton, Calif.). 50 images were collected for each sample; viability was determined by trypan blue exclusion. The cell count and viability of cells injected using the cell therapy injection device was compared to those of uninjected cells.

For the proliferation assay, following injection, Matrigel (BD Biosciences, San Jose, Calif.)-coated plates containing 50 μL 2× NeuroCult NSC proliferation medium (StemCell Technologies, Vancouver, Canada) were seeded with 50,000 cells/well in a volume of 50 μL of basal medium. Uninjected cells were also plated to ascertain any effect of the injection procedure on the cells. A 3H-TdR (tritiated thymidine incorporation) assay was conducted on each plate as follows. A 40 μCi/ml stock solution of 3H-TdR was prepared in the appropriate sterile medium and 25 μl was added to each well to give a final concentration of 1 μCi/well. The plate was then returned to a humidified 37° C., 5% CO2 cell culture incubator for approximately 18 hours. Following the 18 hour incubation, 15 μL of saturated sodium chloride was added to each well to lyze the cells prior to harvesting. The cells were harvested immediately following lysis using a semi-automated 96-well Harvester (Brandel, Gaithersburg, Md.), resulting in the radioactive material from the lysed cells being trapped onto a filter mat. The filter mat was then dried and sealed in a plastic cover with 6 ml of scintillation fluid. Each sealed filter mat was then placed in a cassette and counted in a MicroBeta liquid scintillation and luminescence counter (PerkinElmer, Turku, Finland).

For the apoptosis assay, cells were seeded into 96-well plates exactly as described for the proliferation assay. 100 μL of 2× the final concentration required of staurosporine stocks were then added to the appropriate wells. 18 hours following staurosporine addition, a Caspase-Glo™ 3/7 assay (Promega Corporation, Madison, Wis.) was carried out exactly according to the manufacturer's instructions.

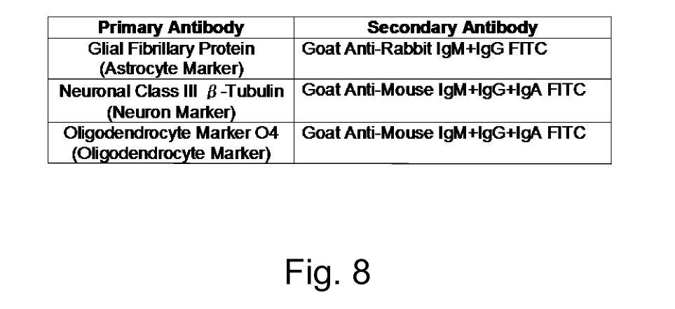

Neurospheres can differentiate into astrocytes, oligodendrocytes, or neurons. For the differentiation assays, following injection, Matrigel-coated plates containing 50 μL 2× NeuroCult NSC differentiation medium (StemCell Technologies, Vancouver, Canada) were seeded with 50,000 cells/well in a volume of 50 μL of basal medium. Uninjected cells were also plated to ascertain any effect of the injection procedure on the differentiation of the cells. The cells were allowed to differentiate for 7 days. Cells were then fixed with 4% paraformaldehyde for 30 minutes at room temperature followed by 3 washes with phosphate buffer solution (PBS). The cell membranes were then permeabilized for 10 minutes at room temperature using a solution of 0.3% Triton X-100 in PBS. Following 2 PBS washes, the cells were labeled with the appropriate primary antibody prepared in PBS containing 10% goat serum for 2 hours at 37° C. as shown in FIG. 8. Following the primary antibody (StemCell Technologies, Vancouver, Canada) incubation, the cells were washed 3 times with PBS prior to adding the secondary antibody (Southern Biotech, Birmingham, Ala.) prepared in PBS containing 2% goat serum as shown in FIG. 8. Cells were incubated with the secondary antibody for 30 minutes at 37° C. Following the 30 minute incubation, the cells were washed 3 times with PBS; distilled water was added to each well following the last wash. Cells were then analyzed on a Synergy 4 Plate Reader (BioTek Instruments, Inc., Winooski, Vt.) and images were captured using a fluorescent microscope.

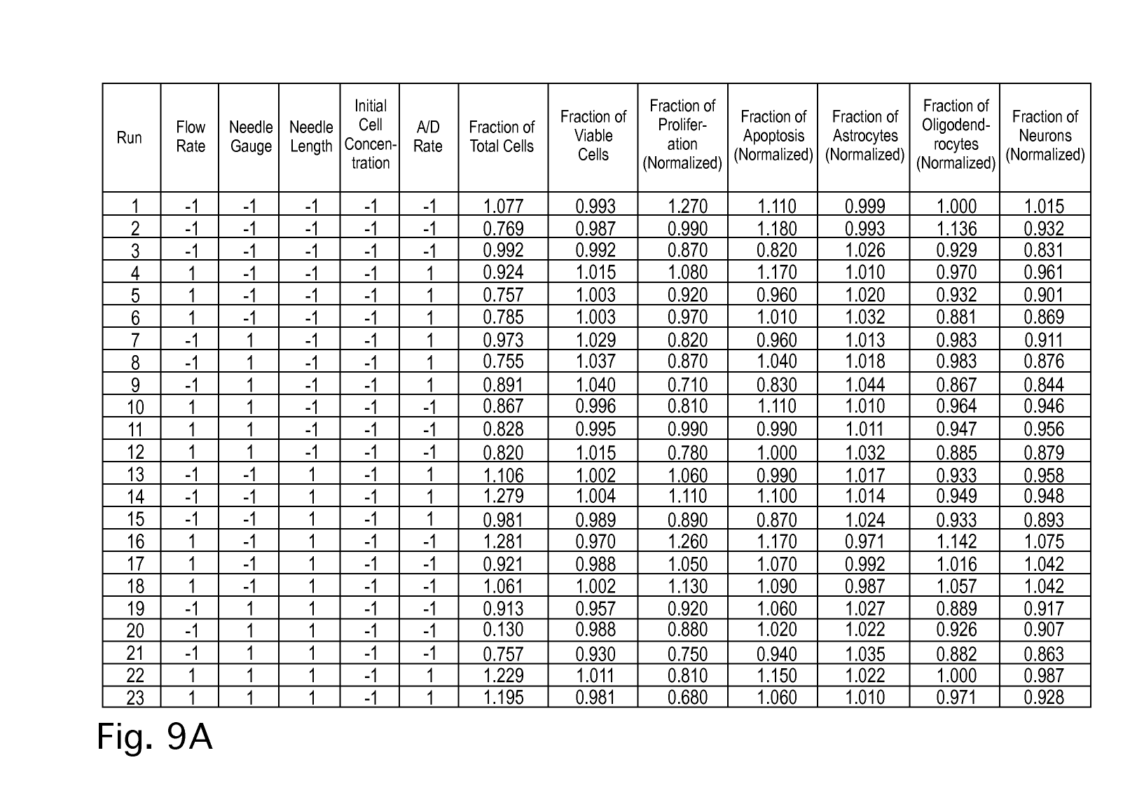

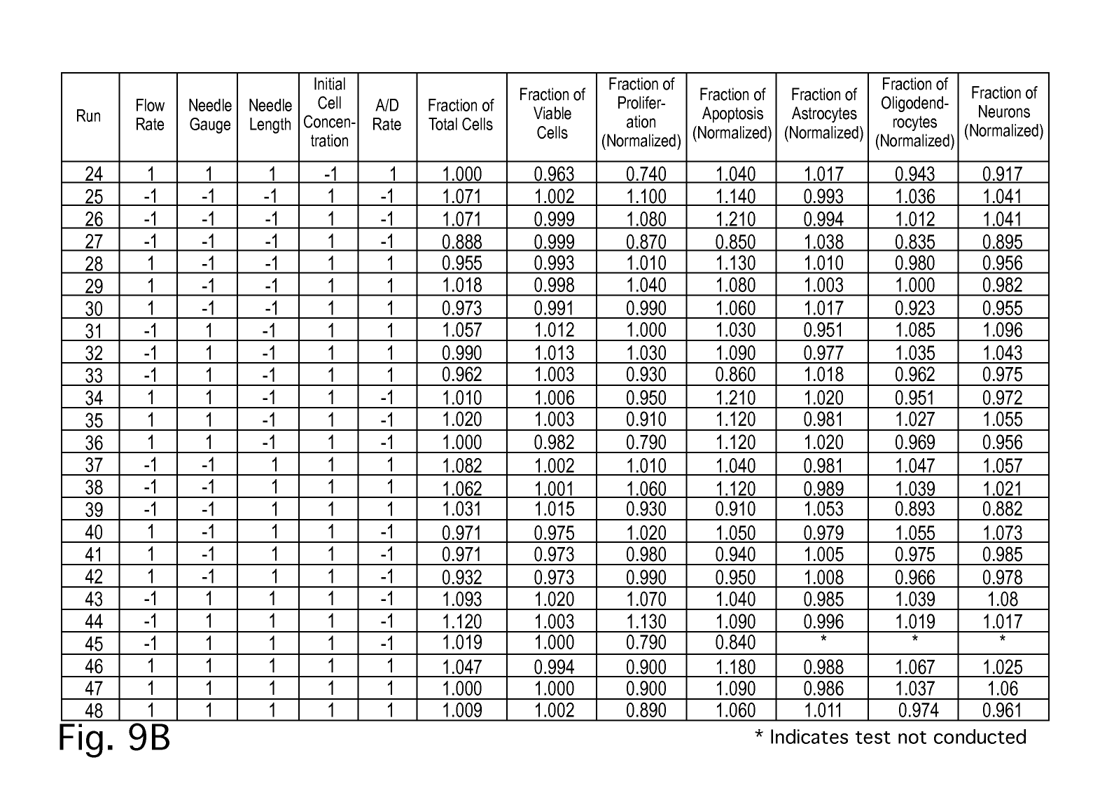

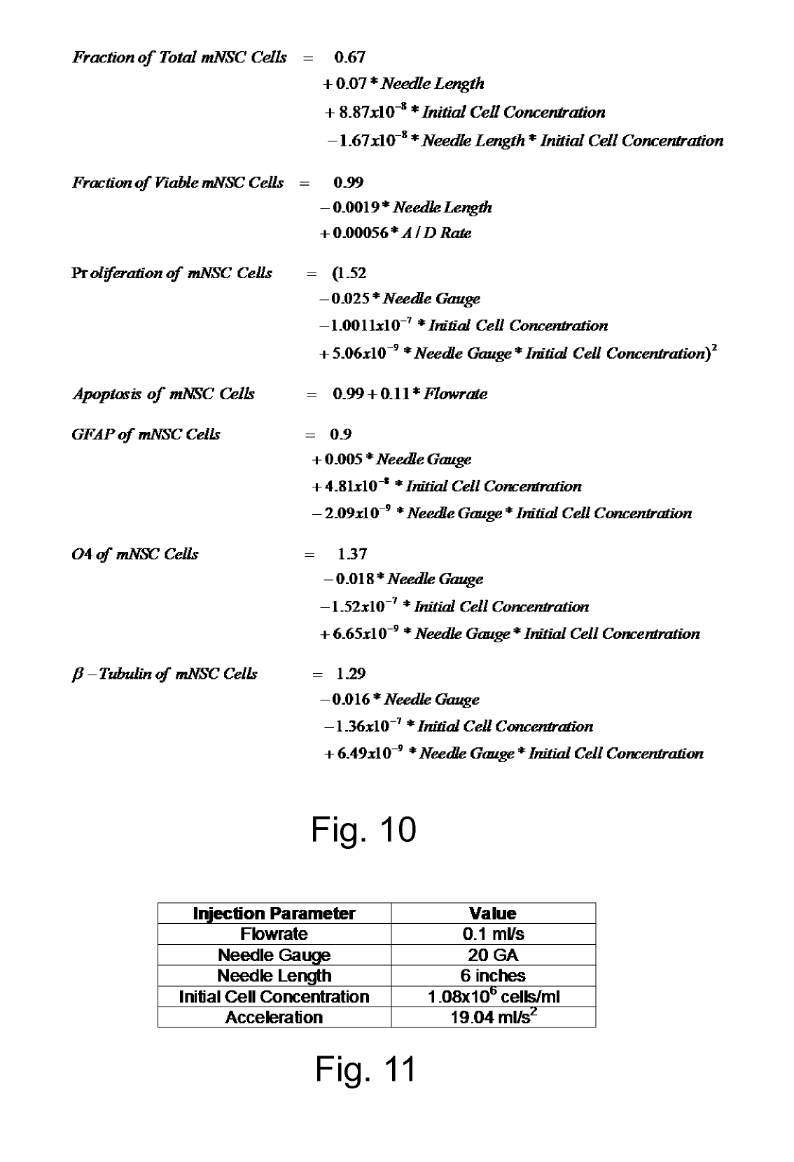

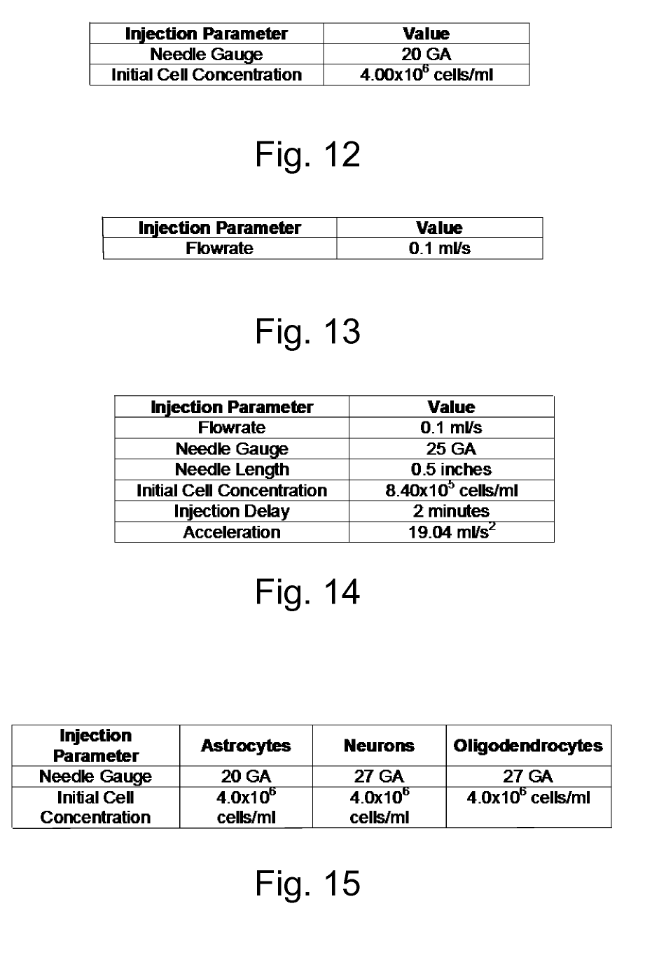

The data from each of the 48 tests is shown in FIGS. 9A and 9B. A “−1” or “1” in the parameter columns indicate either the smallest or largest parameter value, respectively, used for that experimental run. All cellular response data is expressed as a fraction calculated by dividing the specific measured value of the injected cells by the measured value of the uninjected cells. The data was analyzed using MiniTab (MiniTab, Inc., State College, Pa.) experimental design statistical software to develop equations characterizing the relationship between each cellular response and the factors (i.e., injection parameters) upon which it depends. These equations are shown in FIG. 10. The equations were further analyzed using non-linear simultaneous equation analysis techniques to determine the values of each of the injection parameters needed to optimize the combination of all of the cellular responses within the tested ranges of the injection parameters. These values are shown in FIG. 11. These values were entered into a cell therapy injection system such as injection system 10 to allow determination/auto-configuration of injection procedure parameters for mNSC delivery. Using the same technique, injection parameters can be determined to optimize different groupings of cellular response or each cellular response individually, rather than as a group. For instance, FIG. 12 shows the values of injection parameters for optimizing only the proliferation of mNSC, while FIG. 13 shows the values of injection parameters for only minimizing the apoptosis of mNSC. The injection parameter values shown directly affect the indicated cellular responses, while other injection parameters have little or no effect. Moreover, using the same technique, injection parameters can be determined for different cell types or groups of cell types. For instance, FIG. 14 shows the values of injection parameters optimized for the combination of all cellular responses for hCD34+ cells. Note that these values are different from the optimized injection parameters shown in FIG. 11 for mNSC.

Additionally, it was found that by appropriately choosing injection parameters, cells capable of differentiating (for example, stem cells, progenitor cells, blast cells or precursor) could be directed to more or less readily differentiate into specific cell types. For example, FIG. 15 shows the values of injection parameters determined by non-linear simultaneous equation analysis to affect or control differentiation of mNSC into astrocytes, neurons, or oligodendrocytes, respectively. In the studies set forth in FIG. 15, the injection parameter values shown directly affect the indicated cellular responses, while other injection parameters studied were found to have little or no effect.

The foregoing description and accompanying drawings set forth the preferred embodiments of the disclosure at the present time. Various modifications, additions and alternative designs will, of course, become apparent to those skilled in the art in light of the foregoing teachings without departing from the scope of the disclosure. The scope of the disclosure is indicated by the following claims rather than by the foregoing description. All changes and variations that fall within the meaning and range of equivalency of the claims are to be embraced within their scope.

1. A system, comprising:
at least one pressurizing mechanism;
a fluid path adapted to be placed in operative connection with the at least one pressurizing mechanism to deliver an injectate to a patient, the injectate comprising cells;
a control system operably associated with the at least one pressurizing mechanism; and
at least one parameter generation system in operative connection with the control system and comprising an input system to receive data of a type of cells to be injected, the at least one parameter generation system being adapted to generate at least one parameter for an injection procedure at least in part on the basis of the data of the type of cells.

2. The system of claim 1 wherein the at least one parameter for the injection procedure is a variable associated with the injectate, a variable associated with an injection protocol or a variable associated with at least one component of the fluid path.

3. The system of claim 2 wherein the variable associated with the injection protocol is a flow rate, an injection volume, an injection delay, a duration of injection, an acceleration, or a deceleration injection rate.

4. The system of claim 2 wherein the variable associated with the injectate is an initial concentration of cells, a temperature, an agitation or mixing condition, a composition, or an injectate loading condition.

5. The system of claim 2 wherein the variable associated with the at least one component of the fluid path is an identity of the component, a volume, a length, an inner diameter, a material composition, a surface condition, an internal surface coating, or an internal geometrical configuration.

6. The system of claim 5 wherein the at least one component of the fluid path is a needle and the variable associated with the at least one component is a length, an inner diameter, an outer diameter, a curvature, a material composition, a surface condition, an internal surface coating, an internal geometrical configuration, or a number of exit holes.

7. The system of claim 2 wherein the parameter generation system is adapted to be in communicative connection with a memory system, the memory system having stored therein a database of parameters for injection procedures determined at least in part on the basis of the type of cell to be injected.

8. The system of claim 7 wherein the memory system is in communicative connection with the control system.

9. The system of claim 7 wherein the database of parameters for injection procedures comprises parameters determined via at least one optimization study associated with each of a plurality of types of cells.

10. The system of claim 1 wherein the fluid path comprises a container adapted to be placed in operative connection with the at least one pressurizing mechanism so that injectate within the container can be pressurized for delivery.

11. The system of claim 10 wherein the container is a syringe.

12. The system of claim 1 wherein the at least one parameter for the injection procedure is chosen at least in part to control differentiation of a cell that has the ability to differentiate.

13. The system of claim 1 further comprising at least one sensor to identify a fluid path component to be operatively connected to the pressurizing system.

14. An injector system for use in connection with a fluid path to deliver an injectate to a patient wherein the injectate comprises cells, comprising:
at least one pressurizing mechanism adapted to be placed in operative connection with the fluid path;
a control system operably associated with the at least one pressurizing mechanism; and
at least one parameter generation system in operative connection with the control system and comprising an input system to receive data of a type of cells to be injected, the parameter generation system being adapted to generate at least one parameter for the injection procedure at least in part on the basis of the data of the type of cells.

15. A method of injecting an injectate comprising cells in an injection procedure, comprising:
placing a fluid path in operative connection with a pressurizing mechanism to deliver the injectate to a patient; and
generating at least one parameter for the injection procedure at least in part on the basis of the data of the type of cells.

16. The method of claim 15 wherein generating the at least one parameter comprises transmitting the at least one parameter from a database of parameters for injection procedures determined at least in part on the basis of the type of cell to be injected.

17. The method of claim 16 wherein the database of parameters for injection procedures comprises parameters determined via at least one optimization study associated with each of a plurality of types of cells.

18. The method of claim 17 wherein one cell characteristic is optimized or a plurality of cell characteristics are optimized as a group.

19. The method of claim 17 wherein one parameter for the injection procedure is optimized or a plurality of parameters for the injection procedure are optimized as a group.

20. A method of controlling the differentiation of a cell that has the ability to differentiate, comprising: controlling at least one parameter of a fluid comprising the cell or of flow of the fluid comprising the cell through a fluid path.

21. A system, comprising:
at least one pressurizing mechanism;
a fluid path adapted to be placed in operative connection with the at least one pressurizing mechanism to deliver an injectate to a patient, the injectate comprising a biologic;
a control system operably associated with the at least one pressurizing mechanism; and
at least one parameter generation system in operative connection with the control system and comprising an input system to receive data of a type of biologic to be injected, the at least one parameter generation system being adapted to generate at least one parameter for an injection procedure at least in part on the basis of the data of the type of biolgic biologic.

22. A method of injecting an injectate comprising a biologic in an injection procedure, comprising:
placing a fluid path in operative connection with a pressurizing mechanism to deliver the injectate to a patient; and
generating at least one parameter for the injection procedure at least in part on the basis of the data of the type of biologic.
